(19) United States
(12) Patent Application Publication
(10) Pub. No.: US 2005/0096674 A1
(43) Pub. Date: May 5, 2005

(54) Bypass punch anastomosis delivery system

(75) Inventors: Amir Loshakove, Moshav-Bazra (IL); Ido Kilemnik, Herzelia (IL); Ofer Nativ, Rishon-Lezion (IL)

Correspondence Address: William H. Dippert, Esq.;c/o Reed Smith LLP, 29th Floor, 599 Lexington Avenue, New York, NY, 10022-7650, US

(73) Assignee: By-Pass, Inc., Orangeburg, NY (US)

(21) Appl. No.: 10/951,433

(22) Filed: Sep. 27, 2004

Related U.S. Application Data
(63) Continuation-in-part of application No. PCT/IL04/00311, filed on Apr. 4, 2004.
(63) Continuation-in-part of application No. PCT/IL03/00770, filed on Sep. 25, 2003.
(63) Continuation-in-part of application No. PCT/IL03/00769, filed on Sep. 25, 2003.
(60) Provisional application No. 60/492,998, filed on Aug. 7, 2003.
(60) Provisional application No. 60/561,092, filed on Apr. 8, 2004.
(60) Provisional application No. 60/561,091, filed on Apr. 8, 2004.
(60) Provisional application No. 60/518,677, filed on Nov. 12, 2003.
(60) Provisional application No. 60/505,946, filed on Sep. 25, 2003.

(51) Int. Cl. A61B 17/08

(52) U.S. Cl. 606/153

(57) ABSTRACT

An anastomosis and punch delivery system, comprising: a punch section containing a punch mechanism; a connector section containing a connector; and a switching mechanism which pulls said punch mechanism back and replaces it with said connector mechanism.

### RELATED APPLICATIONS

This application is a continuation-in-part of PCT/IL2004/000311 filed on Apr. 4, 2004, which designates the US and which claims the benefit under 119(e) of U.S. Ser. No. 60/492,998, filed on Aug. 7, 2003. This application is also a continuation-in-part of PCT/IL03/00769 filed on Sep. 25, 2003, published as WO 2004/028377, and PCT/IL03/00770 filed on Sep. 25, 2003, published as WO 2004/028376, both of which designate the US. This application also claims the benefit under 119(e) of U.S. Ser. No. 60/561,092 filed on Apr. 8, 2004, U.S. Ser. No. 60/561,091 filed on Apr. 8, 2004, U.S. Ser. No. 60/518,677, filed on Nov. 12, 2003 and U.S. Ser. No. 60/505,946 filed on Sep. 25, 2003. The disclosures of these applications are incorporated herein by reference.

### FIELD OF THE INVENTION

The present invention relates to surgical devices, for example for performing anastomosis connections and/or apertures in blood vessels.

### BACKGROUND OF THE INVENTION

Two blood vessels can be connected to form an anastomotic connection in many methods, including, for example, using surgical clips, using sutures, and using anastomotic connectors, for example as provided by Kaster in U.S. Pat. No. 5,234,447, the disclosure of which is incorporated herein by reference.

### SUMMARY OF THE INVENTION

An aspect of some embodiments of the invention relates to a connector delivery system which includes a mechanism that switches a punch section of the delivery system with a connector section, at an interface location with a blood vessel. In some embodiments of the invention, the switch of the punch section and the connector section is performed automatically after the punch is completed. In some embodiments of the invention, the switch of the punch section and the connector section is performed responsive to a human command which does not directly perform the switch, for example, release of stored power, or pressing a button. In some embodiments of the invention, the switch is manually powered and/or activated. In an exemplary embodiment of the invention, the switch is performed by a single control so that a user can perform the switch fast and correctly. Optionally, the switch is performed fast enough to prevent considerable blood loss. Thus, in some embodiments, there is no need for sealing of the connection of the blood vessel to the delivery system during the switch.

In an exemplary embodiment of the invention, the delivery system guides the connector to fit into a hole made by the punch. Optionally, the hole is maintained by an overtube, which is optionally used for punching.

In an exemplary embodiment of the invention, a connector delivery system provides an all-in-one operation of an anastomosis procedure in that once the system is positioned adjacent a blood vessel (or a tip penetrating a blood vessel), an operator is only required to manipulate one or more controls, until the anastomosis is completed. Optionally, the system itself (e.g., the handle held by the operator) does not need to be moved and/or rotated by the operator, once located adjacent a blood vessel and operated. In some embodiments, some or all of the punching operation requires manual manipulation of the delivery system, for example rotation and/or pushing. Optionally, an operator is only required to penetrate the blood vessel with a sharp tip and once the system is sufficiently advanced, it can operate automatically (optionally with approval from the operator for one or more steps).

An aspect of some embodiments of the invention relates to using a single user control and/or power element for both punch and anastomosis. In some embodiments of the invention, a single handle is used to control both the punch and the anastomosis. In an exemplary embodiment of the invention, replacing of a punch by a connector is provided by shortening a handle. Deploying of the connector is provided by rotating the handle. Optionally, the deploying includes both forward and backwards motion of parts of the connector, relative to the handle. In an alternative embodiment, a button (e.g., to power an electric motor) is activated by a user to advance one stage of the punching and anastomosis process at a time, with each depression performing one stage, such as penetration, cutting, replacing, delivery, deployment and tearing.

An aspect of some embodiments of the invention relates to a punch and connector switching mechanism. In an exemplary embodiment of the invention, the punch is retracted and/or moved out of an axial path of a connector. Alternatively, the punch is retracted and the connector is moved along a non-axial path. Optionally, part of the punch remains and serves as a guiding overtube for the connector.

An aspect of some embodiments of the invention relates to retracting an overtube of a punch or graft delivery system such that the overtube splits before an anastomotic connector is completely deployed. In an exemplary embodiment of the invention, the tube has a tip having a certain diameter and is pulled over a tube or ring having a greater diameter. In an exemplary embodiment of the invention, the overtube is pre-scored. Alternatively or additionally, the overtube is formed of a material with longitudinal fibers, so that tearing is preferentially longitudinal. Optionally, the overtube is retracted before or during the beginning of retraction of forward connector legs.

An aspect of some embodiments of the invention relates to a mechanism for controlling the deployment of an anastomosis connector, including both forward and backwards motion of the connector or portions thereof. The deployment, including both the forward and backward motion, are performed by a human movement in a single direction (e.g., with no reversing) and/or providing a simple control. In an exemplary embodiment of the invention, the deployment control mechanism includes a threaded shaft having a first portion threaded in a first direction and a second portion rotated in a second direction. Optionally, a user (or other power source) rotates or advances the shaft, resulting in both forward and backward movements, sequentially. Alternatively or additionally, nuts mounted on the shaft have opposite threading directions. When rotated, the shaft pulls back a connector and also advances connector spikes to lock on the connector. Optionally, the shaft controls an amount of motion of the different parts and/or tearing of a connector, so that a user is not required to move the delivery system.

An aspect of some embodiments of the invention relates to a self retracting punch. In an exemplary embodiment of the invention, the punch is activated by pulling a pin out of a side of the punch, thereby allowing a spring of the punch to retract at least a portion of the punch. Optionally, the pin is pulled out by the manipulation of a part of the punch compared to another part. For example, trans-axial motion of a penetration tip relative to a cutting tube or motion of a cutting tube (e.g., rotation) relative to a handle or a penetration tip, may release the pin or other mechanism and allow or cause retraction of the punch.

There is thus provided in accordance with an exemplary embodiment of the invention, an anastomosis and punch delivery system, comprising:
a punch section containing a punch mechanism;
a connector section containing a connector; and
a switching mechanism which pulls said punch mechanism back and replaces it with said connector mechanism. Optionally, the system comprises:
a body in which said sections are contained; and
a handle, which when pushed into said body activates said switching. Optionally, rotating said handle deploys said connector and completes an anastomosis connection.

In an exemplary embodiment of the invention, mechanism comprises at least one wire which pulls said punch section.

In an exemplary embodiment of the invention, mechanism is automatic.

In an exemplary embodiment of the invention, mechanism couples said pulling and said replacing.

In an exemplary embodiment of the invention, said switch mechanism is man-powered.

In an exemplary embodiment of the invention, said switch mechanism is machine powered.

In an exemplary embodiment of the invention, said mechanism comprises a synchronizing mechanism, for synchronizing actions of the system.

In an exemplary embodiment of the invention, said system is splittable.

In an exemplary embodiment of the invention, said system is adapted to deploy a connector without the system being moved by an operator relative to a blood vessel.

In an exemplary embodiment of the invention, said switch mechanism moves at least one of said sections along a non-linear path.

In an exemplary embodiment of the invention, said switch mechanism retracts an overtube such that the overtube is torn.

There is also provided in accordance with an exemplary embodiment of the invention, a connector control mechanism, comprising:
a shaft;
at least two nuts mounted on said shaft; and
at least two extensions coupled to said nuts and extending in a same direction,
wherein rotating said shaft in one direction causes one of said extensions to extend and one of said extensions to retract relative to said shaft.

There is also provided in accordance with an exemplary embodiment of the invention, a connector control mechanism, comprising:
a connector;
an overtube adapted to be placed in an aperture of a blood vessel;
a connector advancing mechanism configured to advance said connector through said overtube; and
an overtube retraction mechanism configured to retract said overtube in synchrony with retraction of said connector, prior to full retraction of said connector. Optionally, the mechanism comprises a ring over which said overtube is retracted, said ring having a diameter smaller than that of said overtube.

There is also provided in accordance with an exemplary embodiment of the invention, a punch mechanism, comprising:
a cutting tube;
a penetration tip having an axis;
a spring configured to retract said penetration tip relative to said cutting tube; and
a removable pin provided in a direction perpendicular to said axis and coupling said penetration tip to said cutting tube thereby selectively preventing said spring from said retracting.

There is also provided in accordance with an exemplary embodiment of the invention, a graft delivery system, comprising:
a body;
an overtube adapted to be placed in a blood vessel; an overtube retractor; and
an overtube splitter adapted to split said overtube when said retractor retracts said overtube towards said body. Optionally, said overtube is pre-weakened. Alternatively or additionally, said overtube is pre-split. Alternatively or additionally, said overtube is adapted to act as a blood vessel cutter.

### BRIEF DESCRIPTION OF THE DRAWINGS

Non-limiting embodiments of the invention will be described with reference to the following description of exemplary embodiments, in conjunction with the figures. The figures are generally not shown to scale and any sizes are only meant to be exemplary and not necessarily limiting. In the figures, identical structures, elements or parts that appear in more than one figure are preferably labeled with a same or similar number in all the figures in which they appear, in which:

FIG. 1 is a side view of a delivery system and a loadable graft capsule, in accordance with an exemplary embodiment of the invention;

FIG. 2 is a perspective view of the delivery system with the capsule loaded, in accordance with an exemplary embodiment of the invention;

FIG. 3 is a detail view of the loaded capsule, showing an anastomotic connector, in accordance with an exemplary embodiment of the invention;

FIG. 4 is a detail view of a punch section of the delivery system, in accordance with an exemplary embodiment of the invention;

FIG. 5A is a detail view of the punch section, with a piercing element of the punch retracted, in accordance with an exemplary embodiment of the invention;

FIG. 5B is a cross-sectional view corresponding to FIG. 5A;

FIG. 6 is a view of the delivery system with part of its casing removed, showing the relative placement of the punch and connector, after retraction of the punch section, in accordance with an exemplary embodiment of the invention;

FIG. 7 is a detail view of a handle section of the delivery system, after a bypass catch is released and prior to advancing of the handle, in accordance with an exemplary embodiment of the invention;

FIG. 8 is a detail view of a handle section of the delivery system, after a partial advance of the handle, in accordance with an exemplary embodiment of the invention;

FIG. 9 is a side cross-sectional view of the delivery system, showing the punch section shifted to a side, to allow the connector section to bypass it, in accordance with an exemplary embodiment of the invention;

FIG. 10 is a side cross-sectional view of the delivery system, showing the punch section shifted to a side and fully retracted and the connector section advanced to bypass it and enter into a blood vessel, in accordance with an exemplary embodiment of the invention;

FIG. 11 is a side cross-sectional view of the delivery system, showing an overtube retraction mechanism, in a same state as in FIG. 10, in accordance with an exemplary embodiment of the invention;

FIG. 12A is a side cross-sectional view of the delivery system, showing a partially retracted overtube and partially retracted connector spikes, in accordance with an exemplary embodiment of the invention;

FIG. 12B is a perspective view of the delivery system with part of its cover missing, showing a partially retracted overtube and partially retracted connector spikes, in accordance with an exemplary embodiment of the invention;

FIG. 13A is a perspective view of only a pair of overtube retractors, in accordance with an exemplary embodiment of the invention;

FIG. 13B is a perspective view of the overtube retraction mechanism, in accordance with an exemplary embodiment of the invention;

FIG. 13C is a perspective detail view of the delivery system overtube retraction mechanism, showing the mechanism fully retracting the overtube, in accordance with an exemplary embodiment of the invention;

FIGS. 14A-14D are detail cross-sectional views of a connector deployment in accordance with an exemplary embodiment of the invention;

FIG. 14E is a side view of a connector section of the delivery system, with the cover missing, showing a partial deployed connector, in accordance with an exemplary embodiment of the invention;

FIG. 15A is a side cross-sectional view of a completed anastomosis, in accordance with an exemplary embodiment of the invention;

FIG. 15B is a side perspective view of an anastomotic connector in accordance with an exemplary embodiment of the invention;

FIG. 16 is a flowchart of a process of using the delivery system, in accordance with an exemplary embodiment of the invention;

FIGS. 17A-17G show the retractable handle of the delivery system, in various states, in accordance with an exemplary embodiment of the invention;

FIG. 18 shows a leg confiner element, in accordance with an exemplary embodiment of the invention;

FIG. 19 shows a wire based delivery system, in accordance with an exemplary embodiment of the invention; and

FIG. 20 shows a fluid based delivery system, in accordance with an exemplary embodiment of the invention.

### DETAILED DESCRIPTION OF EXEMPLARY EMBODIMENTS

General Overview

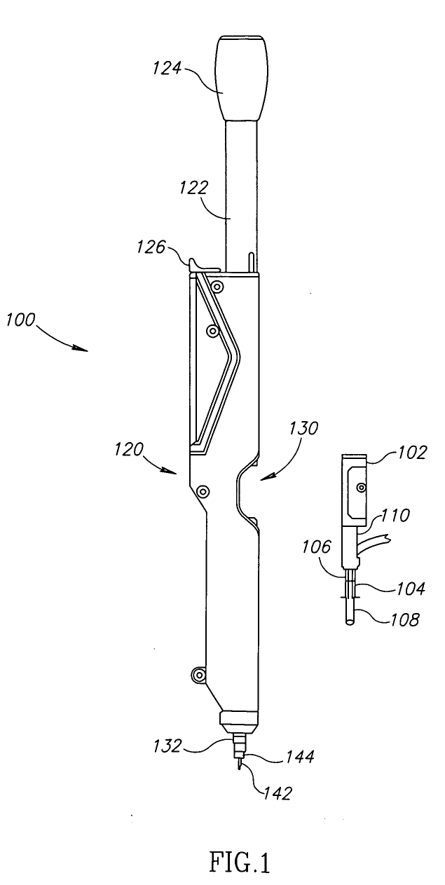

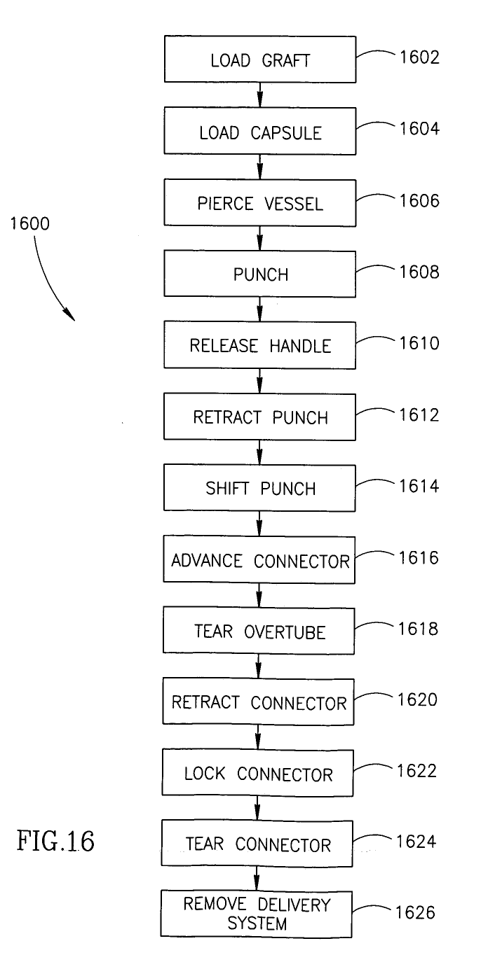

FIGS. 1-15 and 17A-18 show a bypass delivery system 100, in accordance with an exemplary embodiment of the invention. FIG. 16 is a flowchart of an exemplary method of using delivery system 100. Individual ones of FIGS. 1-15 show details of delivery system 100 and/or its configuration in various states of use, generally following the flowchart of FIG. 16. A particular feature of the main illustrated delivery system is that both a punch section and a connector section are provided in the same system and that after punching, the punch section is shifted aside so that the connector section can be brought to bear. Variations on this method and embodiments which do not include both section types (i.e., both punch and connection) are also within the scope of the invention, for example as will be described below. For example, a self-triggering punch mechanism and a tearable overtube can also be provided in a one-section system, which includes only a punch or only a connector deployment mechanism.

In general, the process shown in FIG. 16 comprises setting up the delivery system, forming an opening in a target vessel, replacing the punch section with the connector section, deploying the connector and activating the connector to complete the anastomosis. The section headings generally follow FIG. 16.

Load Graft (1602)

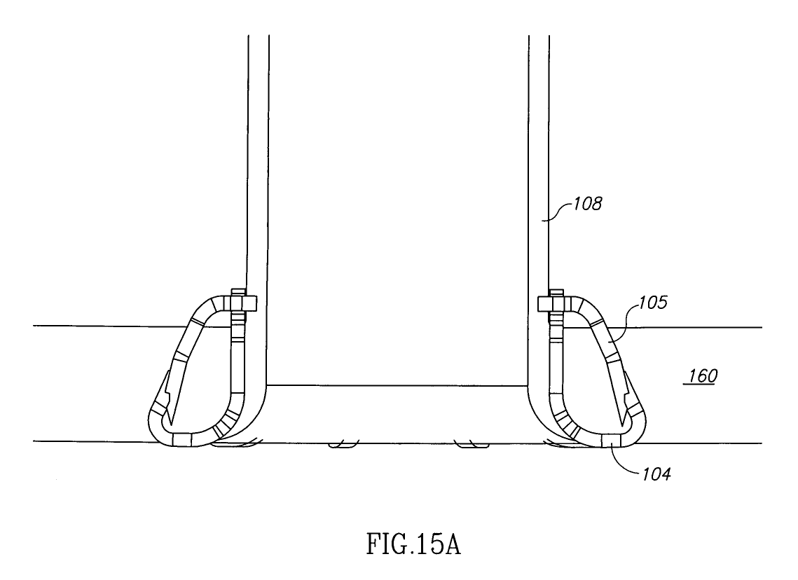

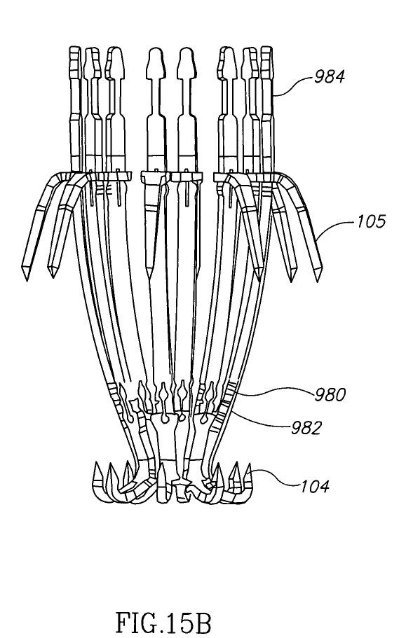

FIG. 1 shows delivery system 100 with a connector section 102 being provided as a separate capsule. A plurality of forward legs 104 of a connector 106 are shown in connector section 102. It should be appreciated that system 100 may be configured for use with a wide range of connectors as is described below. However, a single connector, of the type shown in FIG. 15, is used in many of the figures. This connector comprises a plurality of rail legs which each end in a curved tip, the legs being adapted to have a sliding leg (also with a curved tip) ride along the leg and lock to it in a position where the two curved tips would engage one or more layers of tissue between them. The rail legs are then torn off, so all that remains in the anastomosis is pairs of interlocked curved tip pairs, each pair being independent of the others and forming a staple-like clip.

In FIG. 1, a graft 108 is shown inserted through an opening 110 in a side of connector section 102. This act is represented by 102 in FIG. 16. Optionally, connector section 102 is splitable, for example for easy removal after the anastomosis connection is made. Graft 108 is optionally everted over forward legs 104. However, this is not essential and is not shown, for clarity of presentation. Various methods may be used to load graft 108 into connector section 102. In an exemplary embodiment of the invention, a snare as described in PCT/IL03/00773, mentioned below, is used. Alternatively or additionally, a snare as described in WO 01/70118, the disclosure of which is incorporated by reference, is used. Alternative, for example as described in PCT/IL03/00769, mentioned below, capsule 102 is split, graft 108 is placed into it and capsule 102 is then closed and mounted into system 100. Optionally, capsule 102 is connected to system 100, for example, by a hinge or using a wire as a safety tether.

FIG. 1 also shows other parts of system 100, a body 120, a shortening handle 122 adapted to retract into body 120 and a rotating knob 124 on the handle. An optional locking latch 126 is provided to selectively allow shortening handle 122 to be shortened (e.g., by being pushed into body 120). While the particular mechanism described below in the figures uses a shortening handle and a rotating knob, it should be appreciated that other types of controls can be used to operate system 100. For example, electric, hydraulic or pneumatic motors may be used. For example, an electric motor and battery may be provided in knob 124 and serve to shorten handle 122 and then rotate knob 124, using a suitable gear mechanism. An exemplary hydraulic mechanism which can be used to advance, retract and/or rotate elements is shown in a U.S. provisional application No. 60/561,091 filed on Apr. 8, 2004, the disclosure of which is incorporated herein by reference. Optionally, handle 122 is pre-disposed to shorten and/or rotate, for example, handle 122 including a wound or windable spring, which once handle 122 is released, causes handle 122 to shorten and/or turn.

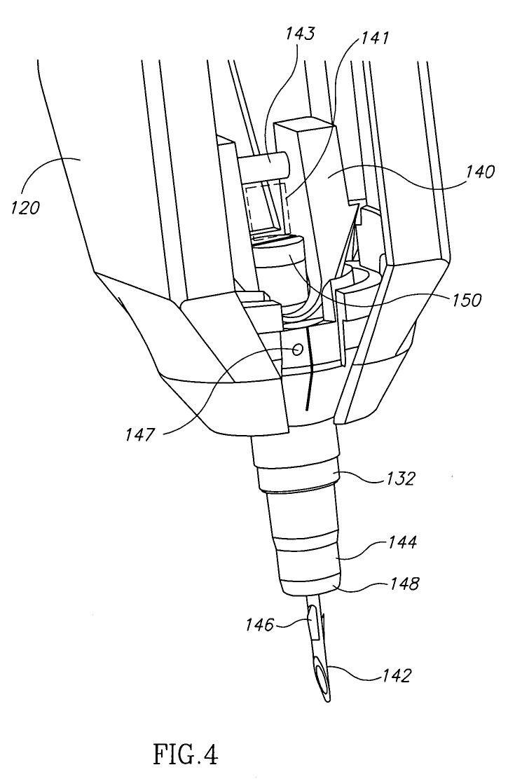

Connector section 102 is fit into a recess 130 of body 120. However, other designs of system 100 may use other insertion locations. At a front end of body 120, can be seen an overtube 132 through which parts of a punch section 140 (shown in FIGS. 4-6 in greater detail) extend. In FIG. 1, a penetration tip 142 and a cutting tube 144 are shown extending through overtube 132.

Load Capsule (1604)

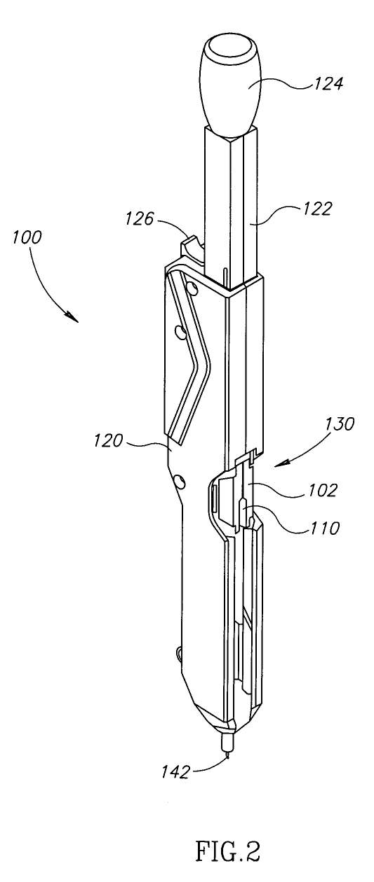

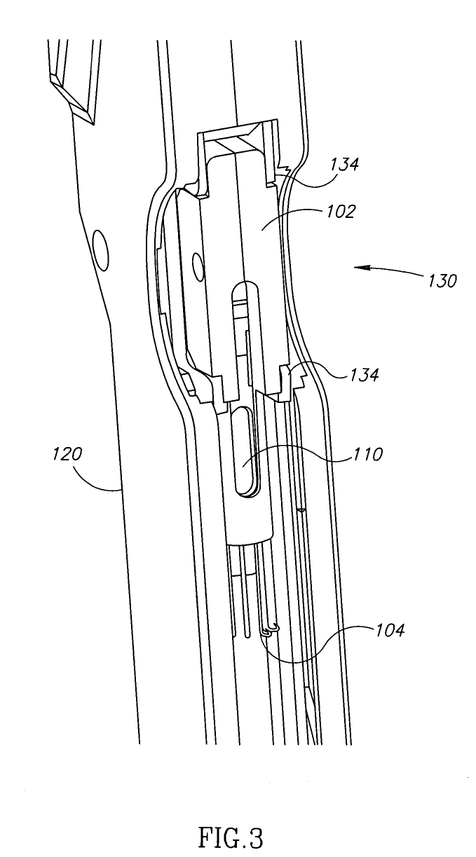

FIGS. 2 and 3 show connector section 102 mounted in system 100. FIG. 2 is a general side view and FIG. 3 is a detail view of the part near recess 130. FIG. 3 also shows an exemplary holding mechanism 134 into which connector section 102 snaps. Other holding methods may be used as well, for example, friction based and adhesive based. Also, as noted above, section 102 may be connected to holding mechanism 134 via a hinge.

Pierce Vessel (1606)

FIG. 4 is a detail section of the front of body 120, showing punch section 140 prior to insertion into a vessel. It should be noted that in some embodiments of the invention a punch mechanism other than that shown is used. An application list provided below includes applications that show various punch mechanisms and/or connectors which may be used in association with a delivery system in accordance with exemplary embodiments of the invention.

In the punch design shown, a penetration tip 142 is sharp and hollow and includes one or more barbs 146 which open out and engage the wall of the blood vessel from inside the blood vessel after penetration tip enters the blood vessel. Barbs 146 optionally serve to retract the vessel wall and/or to prevent a punched out portion from falling into the blood stream. Cutting tube 144 includes a forward cutting edge 148 adapted to cut through blood vessel wall. Cutting tube 144 optionally has a stepped or inclined increasing outside diameter to assist in conveying the blood vessel wall to lie over overtube 132, as will be described below.

Punch (1608)

After penetration, the punching of an aperture in the target vessel is achieved by advancing system 100 so that cutting edge 148 contacts the blood vessel wall. Optional rotation of system 100 can assist the cutting action. In an exemplary embodiment of the invention, penetration tip 142 is retracted, pulling back with it barbs 146 and thereby retracting the vessel wall against cutting edge 148. Optionally, cutting edge 148 is spring-loaded to rotate, once released to do so, for example by retraction of penetration tip 142 releasing a holding pin.

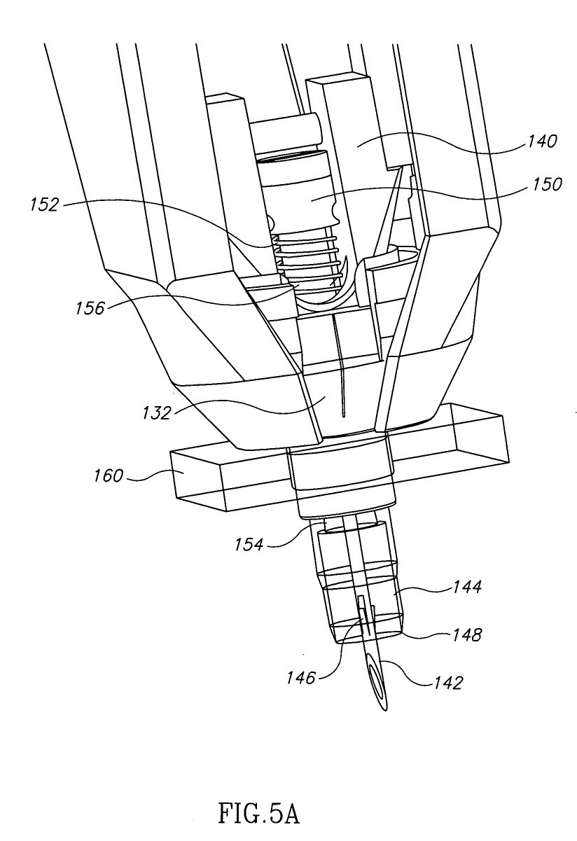

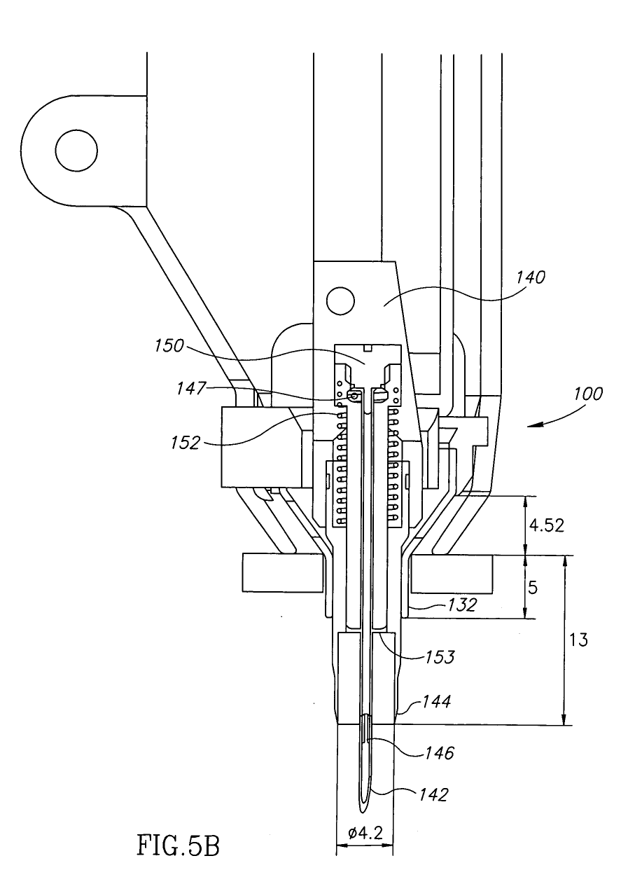

FIG. 5A shows penetration tip 142 retracted. In an exemplary embodiment of the invention, retraction is manual. In some exemplary embodiments, a blocking pin 141 is used. In one example, pin 141 (shown in FIG. 4) is provided between base 150 and a section 143 of punch section 140. This pin prevents a spring 152 (described below) from releasing. In another embodiment, an aperture 147 is provided through punch section 140 and system 100, so that pin 141 (of a smaller size) can lock the retracting section of punch 140 to the body. Optionally, a trigger mechanism is used to automatically retract penetration tip 142 after the wall of the target blood vessel is sufficiently penetrated. An example trigger mechanism is shown in U.S. provisional application 60/492,998, thereafter filed in the PCT on Apr. 4, 2004 as PCT/IL2004/000311, the disclosures of which are incorporated herein by reference. In one embodiment shown in this application, a trigger tube 154 is provided (shown in FIG. 5 already retracted) forward of cutting edge 148 and optionally surrounding penetration tip 142, at a section proximal of barbs 146. When penetration tip 142 is inserted into a vessel, once the insertion is deep enough, the blood vessel wall contacts and pushes back trigger tube 154. Trigger tube 154 is set up to release a latch that maintains a base 150 in a locked position. Once unlocked, a compressed spring 152 expands and pulls back base 150. Base 150 is coupled to penetration tip 142 (and optionally trigger 154) so that penetration tip 142 is retracted with the movement of base 150. Optionally, an oil-filled cylinder 156 is provided to slow down the retraction process. An exemplary locking mechanism released by trigger 154 is as follows. An inner shaft (not shown) is locked to a tube extension of base 150 by one or more balls. Trigger 154 includes one or more openings which when moved adjacent the balls allow the balls to move through the openings and release the locking of base 150. Other mechanisms may be used as well. For example, instead of a separate trigger tube, cutting tube 144 may itself act as a trigger when pushed against the blood vessel wall. In one example, relative sideways motion of penetration tip 142 and cutting tube 144, releases such balls or pin 141 (which may be spring loaded to retract out of hole 147). An optional safety mechanism is a cap on the punch section which maintains the relative axial positions of tip 142 and tube 144.

After cutting edge 148 completes cutting a hole in the target blood vessel, edge 148 is further advanced into the blood vessel. As noted above, tube 142 may have an increasing outer diameter, so that advancing the blood vessel wall onto overtube 132 encounters no sudden large changes in geometry. A potential advantage is that the punched hole may be made smaller than the final diameter of the anastomosis, thus applying pressure on the graft (when attached). Another potential advantage is that a larger hole is available for providing the connector than the actual diameter of the anastomosis, thus, for example, allowing the use of a non-expanding connector.

FIG. 5B is a side cross-sectional view of the forward end of system 100. An optional seal 153 is provided to prevent cut tissue portions and/or blood from entering into system 100. Alternatively or additionally to being a seal, element 153 may function as a guide for penetration tip 142.

As shown, a section 160 (FIG. 5A) of the blood vessel wall is mounted on overtube 132.

Release Handle (1610)

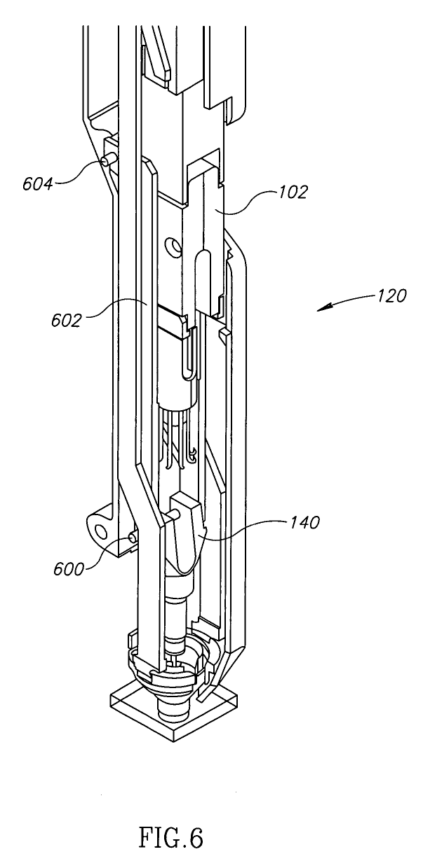

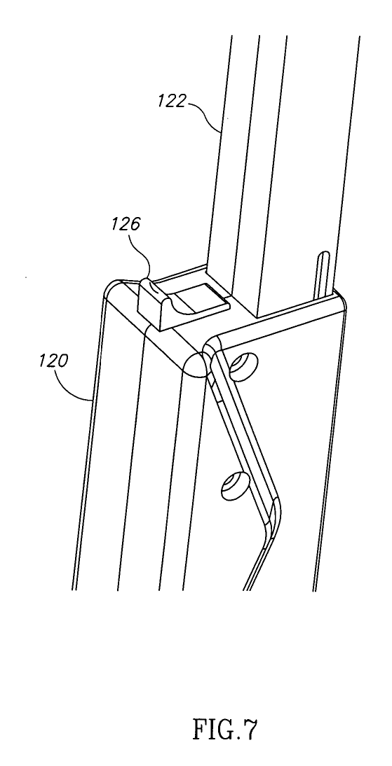

With the punching completed, punch section 140 is moved out of the way and connector section 102 is advanced. FIG. 6 shows system 100 with part of its cover (e.g., body 120) removed and showing a punch retraction mechanism, in a stage where the punch is already partly retracted. In the embodiment shown, the punch is retracted and connector advanced by shortening handle 122 (FIG. 1). FIG. 7 shows details of latch 126, in open position. In an alternative embodiment of the invention, separate handles are provided for advancing the connector section and for retracting the punch section.

Retract Punch (1612)

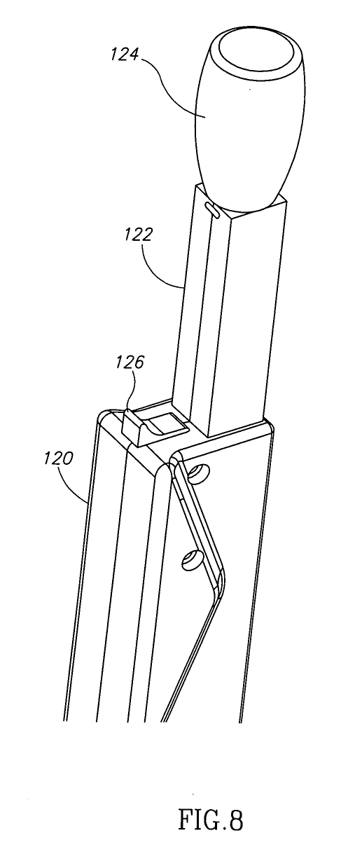

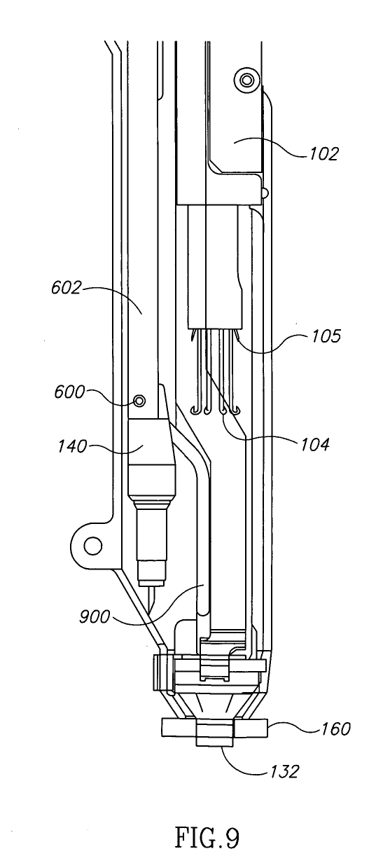

FIG. 8 shows handle 122 partially shortened, and corresponding to FIG. 6, in which punch section 140 is pulled back. Referring back to FIG. 6, punch section 140 is connected to one or more elongate elements 602 which are retracted by the retraction of handle 126 as described below. Flexible elements, such as wires, cables or flexible strips, rather than rigid elements may be used as well. In the mechanism shown, a pin 600 is used to couple elements 602 to punch section 140 and extends so that pin 600 sits in a slot (shown in FIG. 9).

In an embodiment where tube 144 also serves as an overtube, optionally only penetration tip 142 needs to be retracted, which can allow system 100 to be narrower. Optionally, also a triggering mechanism, if any is provided, is pulled back with penetration tip 142.

Shift Punch (1614)

FIG. 9 is a side cross-sectional view of system 100, showing punch section 140 shifted to one side. Pin 600 sits in a slot 900, optionally defined on either side of the cover of body 120. The shape of slot 900 is optionally curved or includes a bend, so that retraction of elements 602 first retracts punch section 140 straight backwards in an axial direction and then shifts section 140 in a trans-axial direction. In the embodiment shown, the trans-axial shift also includes an axial retraction component, but it is not essential, especially if wires or other pliable elements are used instead of rigid elements for elements 602, and can pull the punch section sideways. Thus, punch section 140 is moved out of the way from the axial advance of connector section 102.

Short spikes 105 are the forward tips of sliding sections of the connector, and serve, for example, to engage an outside wall of a side vessel.

Advance Connector (1616)

Continued shortening of handle 122 pushes connector section 102 into the blood vessel, through overtube 132. In one embodiment, shortening of handle 122 is always coupled to movement of connector section 102, and shifting of punch 140 aside is completed before advancing connector section 102 reaches punch 140. In an alternative embodiment, handle 122 engages connector section 102 only after it is shortened enough so that punch section 140 is moved out of the way.

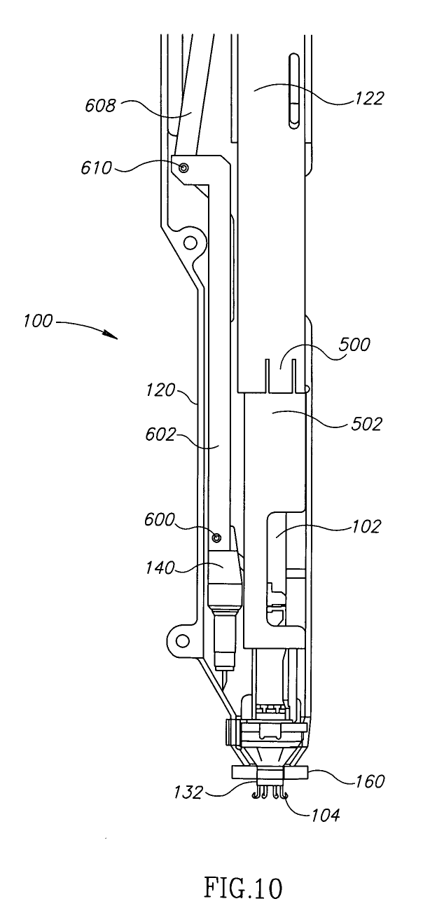

FIG. 10 shows connector section 102 fully advanced. Also shown is an elongate element 608 which is coupled to element 602 by a joint 610, described in FIG. 11. In the embodiment shown, connector section 102 is held in a holder 502 which is engaged by an engagement section 500 of handle 122, when handle 122 is shortened enough.

In an exemplary embodiment of the invention, the replacement of punch section 140 with connector section 102 is fast enough so that any blood leakage from the target vessel is minimal. Alternatively, a valve is provided to prevent leakage of blood during the exchange. In an exemplary embodiment of the invention, a leaflet valve (not shown) is provided inside overtube 132, such that connector legs 104 can open the valve when it is passed through. In some embodiments, the punch section is only retracted by a user and not advanced, so it does not need to penetrate past the valve. Optionally, the valve is mounted on overtube 132 itself.

In an exemplary embodiment of the invention, no valve is provided, as the switching of the punch section by the connector section in some embodiments is fast enough and without an opportunity for a user to pause in the middle of, that only a small leakage of blood is expected. For example, the switch may be completed in less than 10, 5, 3 or 2 seconds. Optionally, the mechanisms of system 100 (e.g., mechanism 700 described below) are at least partially sealed to protect them from blood.

FIG. 11 is a side view of all of system 100, showing handle 122 fully shortened and showing a punch retraction mechanism 700. Some elements are hidden for clarity in this and other figures, for example overtube 132. A pin 604 (FIG. 6) is optionally provided in joint 610 and may optionally serve the purpose of a projection which rides in a groove (e.g. like pin 600), to guide the retraction of punch section 140.

It should be noted that while the design shown shifts punch section 140 aside, in alternative designs, connector section 102 travels along a non-linear path, for example, using a pin (e.g., pin 604) that rides in a non-linear path. This is alternatively or additionally to punch section 140 riding a non-linear path. Also, in some designs, punch section 140 is retracted further, for example, system 100 may have an elongate narrow neck adapted for insertion into the body. Punch section 140 is optionally retracted out of the neck before connector section 102 is passed through the neck. Optionally, the part of system 100 where the sections pass by each other is outside of the body.

In an exemplary embodiment of the invention, overtube 132 functions as a punch cutting edge. Then, only penetration tip 142 needs to be pulled back and moved out of the way. Overtube 132 is optionally pre-slit, optionally with only a thin layer of unsplit and/or unweakened material at its tip, to define a smooth cutting edge. Optionally, a layer of metal is provided at the tip for cutting. Optionally, this layer is pre-split at its proximal end, to match slots that are preformed in overtube 132. Alternatively or additionally, the proximal side may be serrated. Optionally, the un-split portion is only a single wire embedded at a distal end of the overtube. In an alternative embodiment, a split cutting edge 148 is used for cutting. Optionally, the separate parts of overtube 132 abut. Alternatively, the parts may overlap, for example, in the form of overlapping leaves.

Referring to mechanism 700, in an exemplary embodiment of the invention, element 608 is attached to a toothed bar 702, by a joint 706. Handle 122 also includes a toothed section (not shown). A gear 704 couples the two toothed sections so that shortening of handle 122 causes retraction of element 608 and eventually of punch section 140. Optionally, a small gear 708 is provided axially attached to gear 704 and being coupled to shaft 122. Thus, the ratio of motion of element 608 and of handle 122 can be set by the gear ratio.

Latch 126 is optionally spring loaded so that it locks handle 122 in its shortened position.

Tear Overtube (1618)

In some embodiments of the invention, overtube 132 obstructs the completion of the anastomosis, for example, if one or more spikes need to contact the target vessel from the outside. In an exemplary embodiment of the invention, overtube 132 is retracted out of the target blood vessel. In some embodiments of the invention, the inner diameter of overtube 132 is smaller than that required to pass all of connector 106 or the forward portion of connector section 102. In an exemplary embodiment of the invention, overtube 132 is pre-defined to include one or more slits and/or is pulled back over a hard and/or sharp object which tears it.

FIG. 11 shows an overtube holder section 806 which is adapted to engage overtube 132 (not shown) or be contiguous with it. An elongate arm 800 (or as noted above, a wire) is coupled via an optional side segment 804 to a retracting pin 802. In the embodiment shown, a spring coupling 805 is provided between segment 804 and element 800, to allow for pin 802 to push segment 804 out of the way when handle 122 is shortened. Other mechanisms may be used as well.

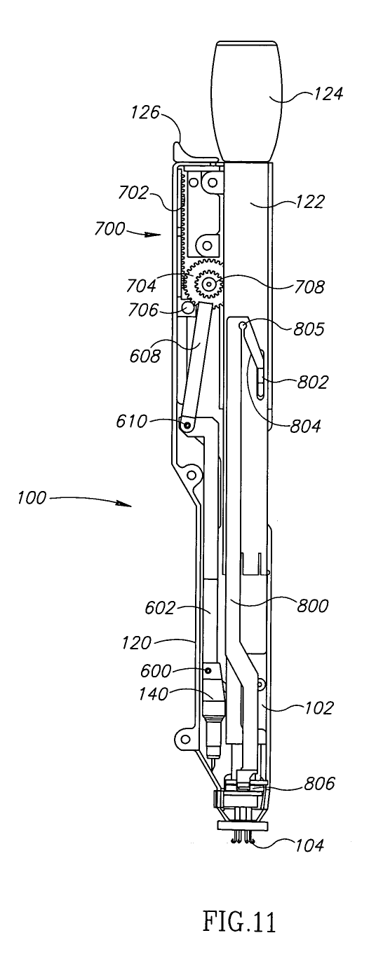

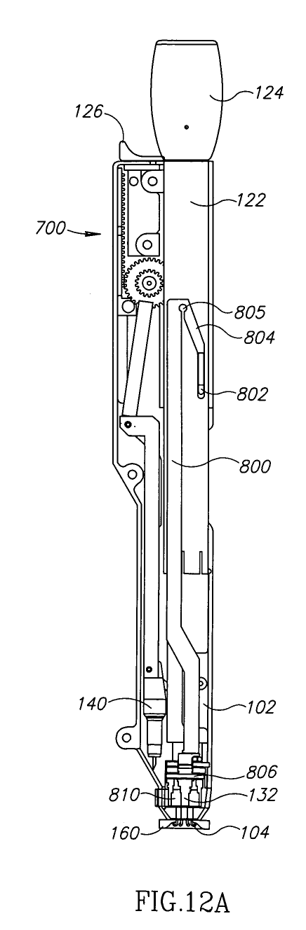

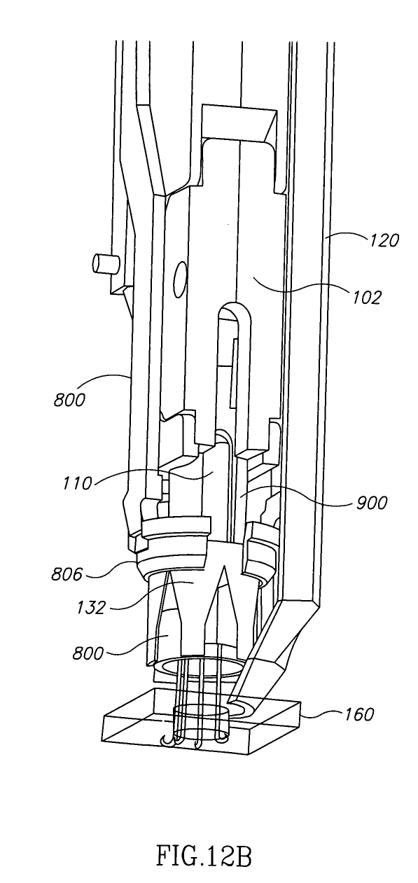

Rotation of handle 124 causes the retraction of pin 802, which also retracts overtube 132, tearing it as shown in FIGS. 12A and 12B. Optionally, this retraction also retracts connector legs 104, for example by retracting connector section 102, so that graft vessel 108 is pulled against vessel wall section 160. In some embodiments of the invention the exact timing of the pulling back of overtube 132 and of the legs is not important. Optionally however, overtube 132 is retracted early enough so that overtube 132 is not in the anastomotic connection when legs 104 start pulling back on the target vessel tissue. In some cases, overtube 132 may interfere with proper engagement of the tissue by legs 104. Alternatively, overtube 132 is used to shape aperture 132 into a desired shape for legs 104 to engage, while they retract.

FIG. 12B is a detail view of the forward end of system 100, showing overtube 132 torn by being pulled over a tube or ring 810.

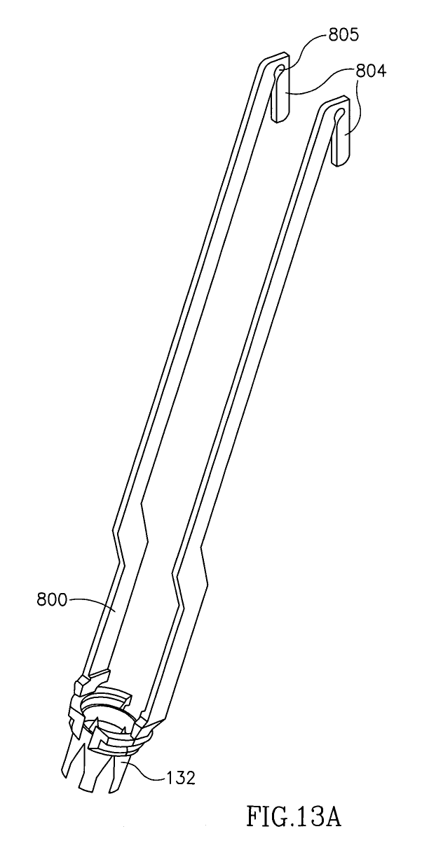

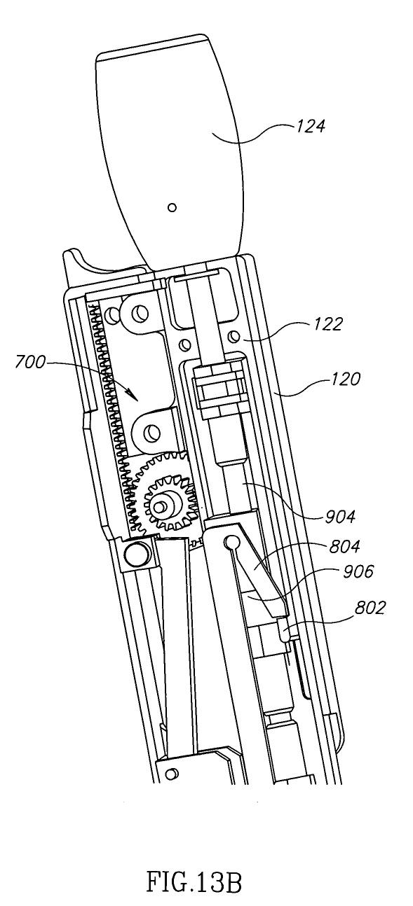

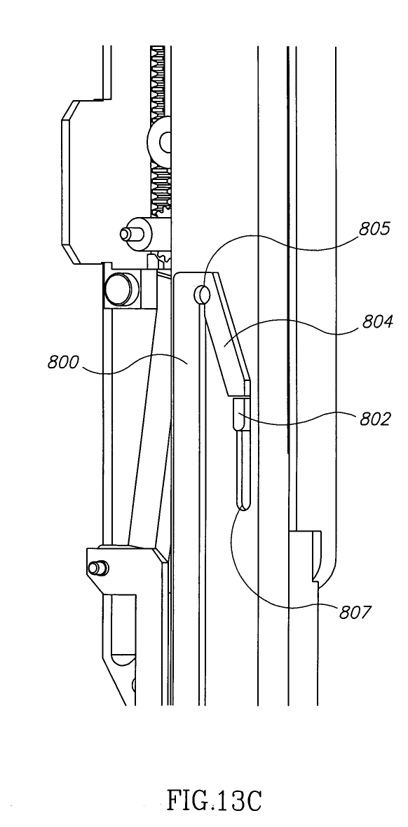

FIG. 13A shows only overtube 132 and its retractor (800 and 804).

FIG. 13B shows mechanism 700 and the inside of handle 122, showing the overtube retraction mechanism. Rotation of knob 124 rotates a shaft 904. In an exemplary embodiment of the invention, shaft 904 is threaded and a plurality of nuts ride on it. A nut 906 is coupled to pin 802 and is prevented from turning by the inner geometry of handle 122. Rotation of shaft 904 causes nut 906 to move towards knob 124, thereby pulling back overtube 132 and tearing it. A similar mechanism is used for pulling back forward legs 104.

FIG. 13C shows pin 802 fully pulled back in a slot 807 defined for it.

In an exemplary embodiment of the invention, overtube 132 is pre-scored to tear in desired locations. Alternatively or additionally, overtube 132 is formed of a material with longitudinal fibers so tearing is longitudinal. In one example, overtube 132 is formed of Polypropylene, with longitudinal fibers, formed, for example, by stretching in a longitudinal direction.

In an exemplary embodiment of the invention, overtube 132 is formed of vacuum-formed polystyrene, which is stretched axially while being formed so that tears propagate in an axial direction. Weakening, for example, slots that penetrate through all or part of the thickness of overtube 132 are optionally provided to initiate such tears. Other axially tearing materials, for example, metals and polymers may be used as well. Optionally, when overtube 132 is used as a cutting tube 144, overtube 132 is made stiff enough to be able to apply force to the blood vessel, while cutting.

Retract Connector (1620)

In an exemplary embodiment of the invention, together with or after tearing of overtube 132, forward legs 104 are retracted. In an exemplary embodiment of the invention, differential retracting rates for overtube 132 and legs 104 are provided by using different threading angles on nut 906 and on the nut (not shown) which retracts legs 104. In an exemplary embodiment of the invention, all of connector section 102 is retracted. Alternatively, only a connector holding section 900 (FIG. 12B) is retracted, into section 102. In an exemplary embodiment of the invention, connector holding section 900 engages forward legs 104 in a manner which will engage it to tear them, as described below.

In an alternative embodiment, nut 906, once it reaches a certain point on shaft 904 engages an extension of section 900, thereby retracting it.

In an alternative embodiment of the invention, separate mechanisms are used for pulling back and tearing overtube 132 and for retracting the connector. In an exemplary embodiment of the invention, separate user-manipulated controls, for example knobs, are provided. Optionally, a simple puller is provided to pull back the overtube. A potential advantage of using separate mechanisms, in this or in delivery-only systems, is that tearing of the overtube may be made less exact than deployment of the connector, which may require a more precise choreography of the motion and tearing.

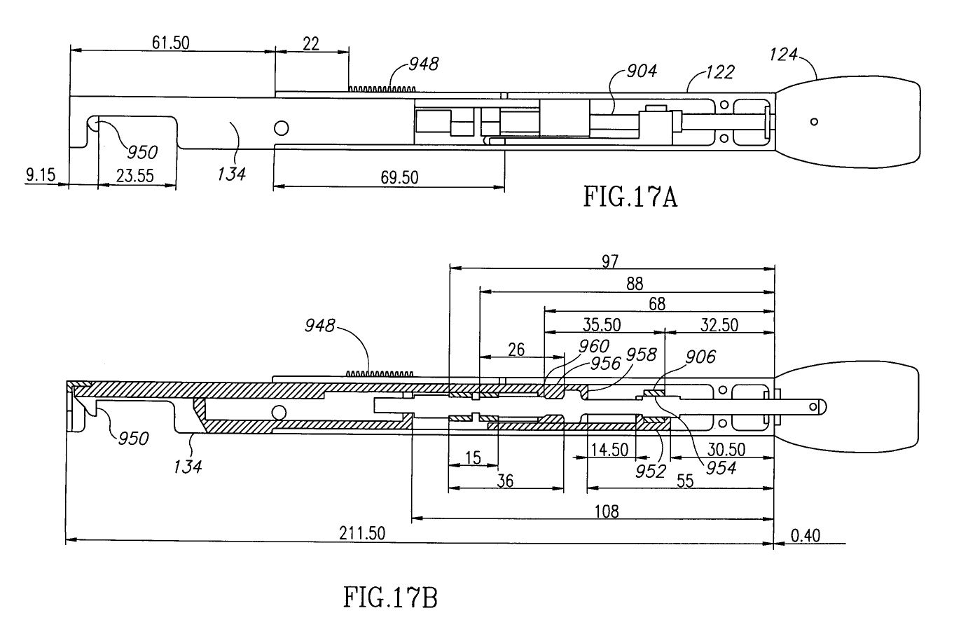

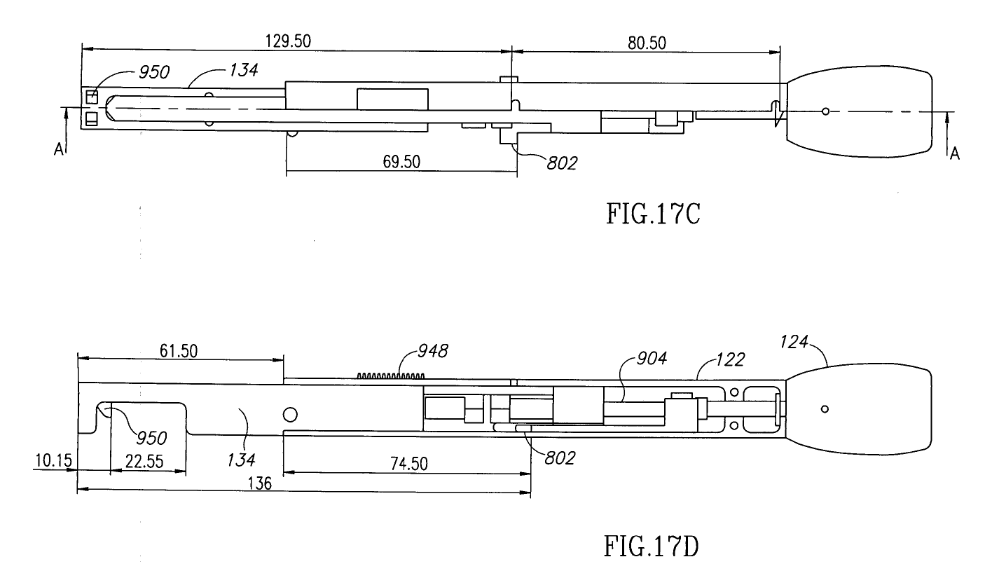

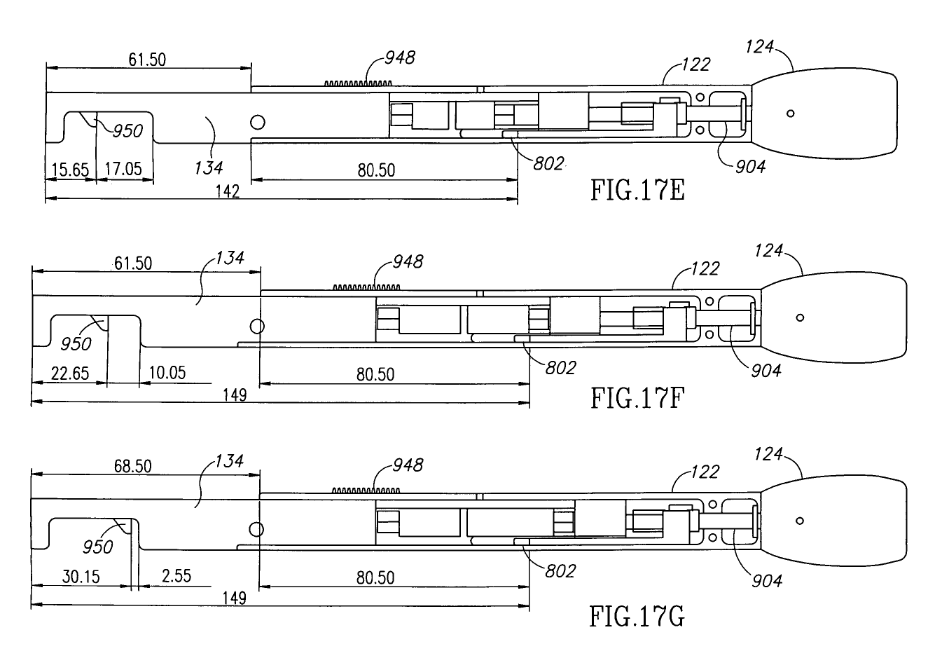

FIGS. 17A-17G show handle 122, in various states, in accordance with an exemplary embodiment of the invention. FIGS. 17A-17C show the handle from various views, prior to retractions. In FIG. 17B, for example, a cross-sectional view, nut 906 is shown mounted on a threaded section 954 of shaft 904. A housing 952 is mounted on nut 906, prevents it from rotating and is moved by nut 906, to retract pin 802. Once the nut threads past section 954, the retraction is stopped. A similar mechanism is optionally provided for each of the retracted and/or advancement steps described herein. A delay between the activation of the various mechanisms is provided, for example, by axially separating the nut from the housing. For example, a nut 956 for retracting a pin 950 is axially separated from an edge 958 of a housing 960 that pulls back pin 950.

An optional notched section 948 interacts with gear 704, as described above.

FIG. 17D shows the state before tearing of overtube 132. In FIG. 17E, the overtube is torn (pin 802 retracted) and a connector retracting pin 950 is also retracted. Pin 950 pulls back tube 900, to retract forward legs 104.

In FIG. 17F, pin 950 is further retracted, but at a same time, holder 134 is advanced, such that backwards legs of the connector can advance and lock the connector. This is described below. The threading for advancing holder 134 is optionally in an opposite pitch from the threading for the other movements.

In FIG. 17G, pin 950 is further retracted, thereby tearing the connector.

In an exemplary embodiment of the invention, the retraction of the connector retracts the target vessel wall inwards from the plane of the blood vessel.

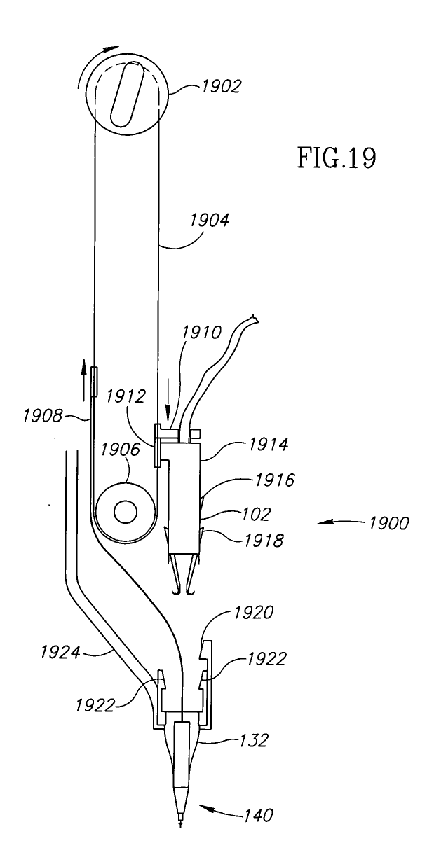

It should be noted that instead of a thread and nut based retraction and timing mechanism, other mechanism can be used. For example, the retracting elements may be pulled by a wire, which wire is selectively taken up by one or more spools connected to shaft 954, as knob 124 is rotated. Optionally, the use of wires for a control mechanism allows to make delivery system 100 flexible, for example suitable for a catheter, a maneuverable laparoscope, or for narrow access surgery such as limited access keyhole surgery. A pulley, for example in the shape of a pin at a front end of system 100, may be used to advance portions of system 100, by pulling on a wire that rests on the pulley and is coupled at its other end to the connecter section. An exemplary wire embodiment is shown in FIG. 19, below.

Lock Connector (1622)

After retracting the forward legs of the connector, the backwards legs of the connector are deployed and locked to the front legs (in the embodiment shown).

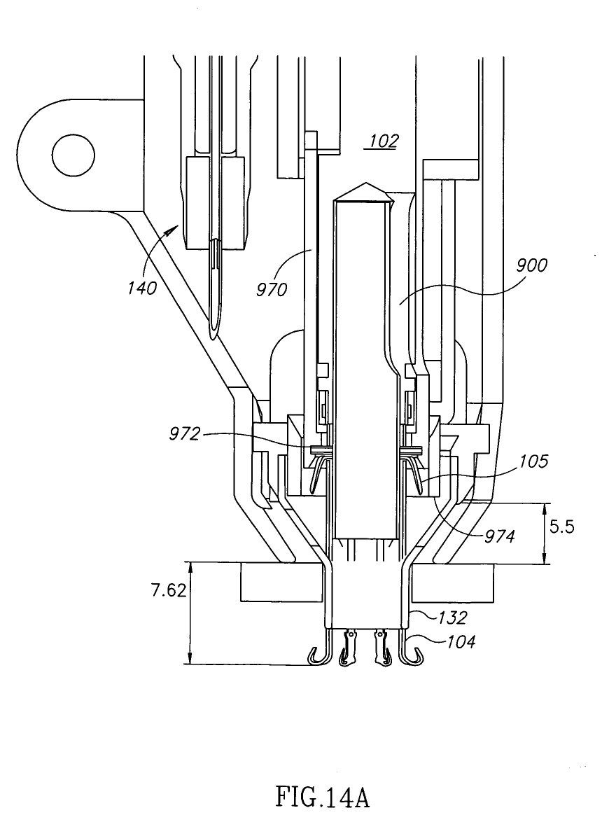

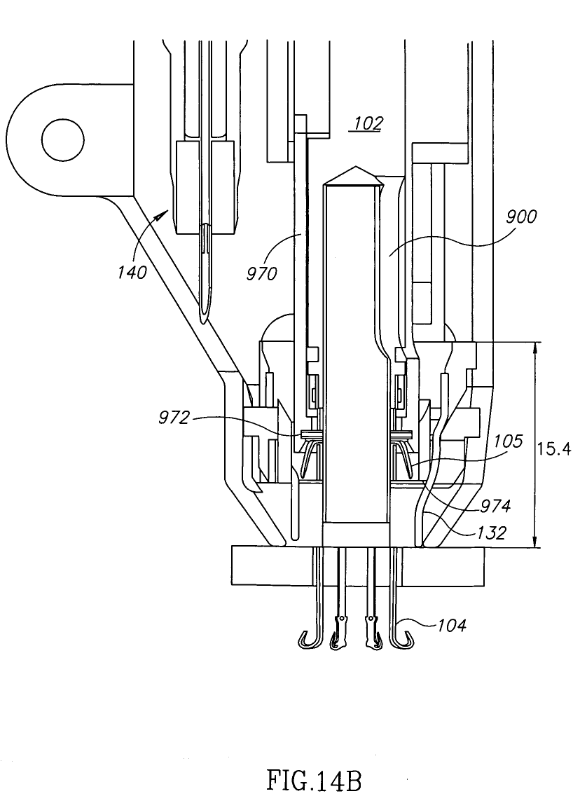

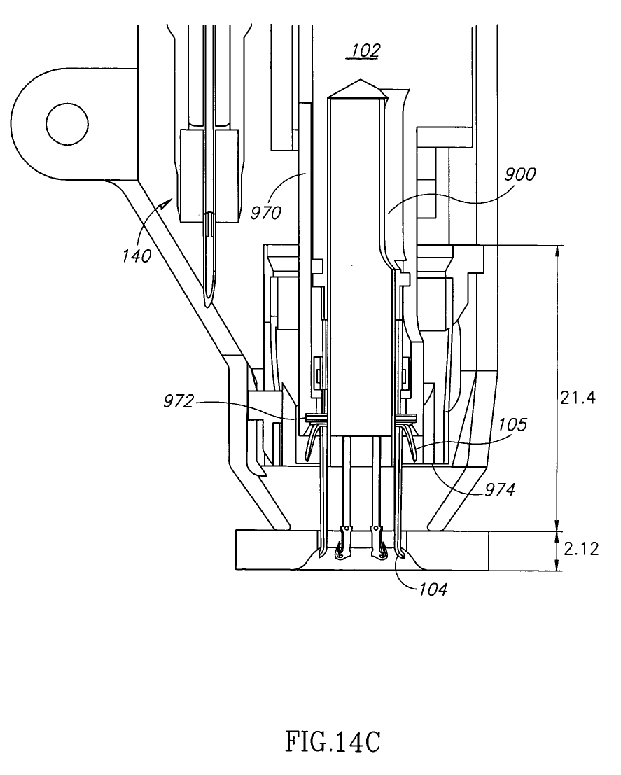

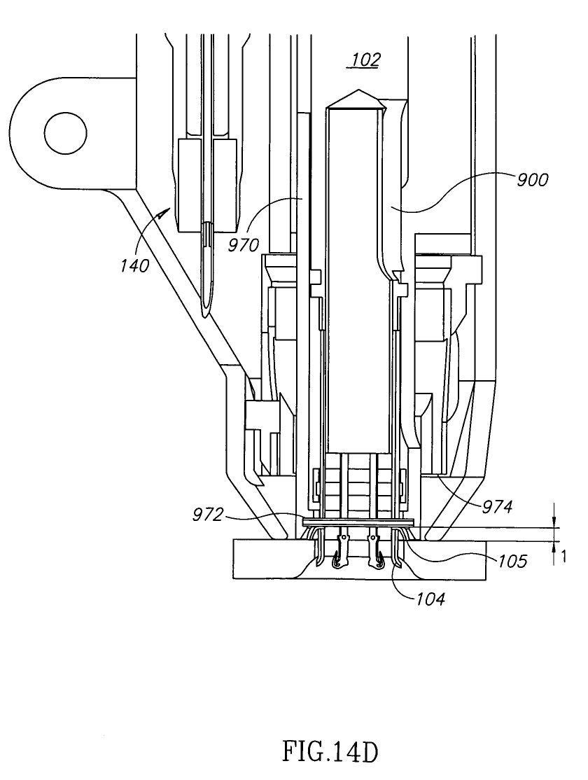

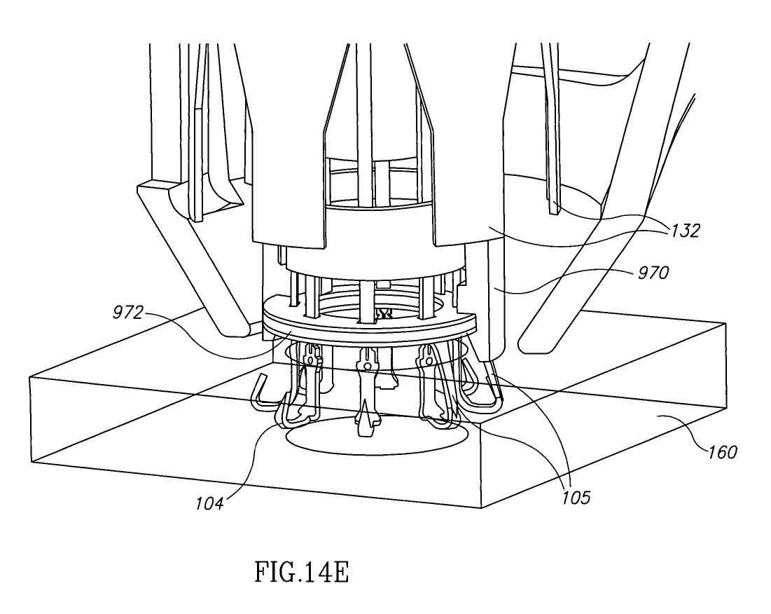

FIGS. 14A-14D are detail cross-sectional views of a connector deployment in accordance with an exemplary embodiment of the invention. FIG. 14A shows the state after forward legs 104 are advanced through overtube 132. A plurality of sliding spikes 105, which serve as the backward legs of the anastomosis connector, rest against a ring 972, which is used to advance spikes 105. A tube 970, is coupled to shaft 904 via a nut and engages ring 972 for advancing and retracting thereof. As in the other figures, graft vessel 108 is not shown, for clarity.

A tube 974 is optionally provided to protect spikes 105 and/or to assist in tearing overtube 132, by retraction of overtube 132 overtube 974.

FIG. 14B shows the layout after the retraction of overtube 132 and prior to retraction of forward legs 104.

FIG. 14C shows retraction of forward legs 104.

FIG. 14D shows the advance of ring 972 such that spikes 105 are advanced to lock to forward legs 104. In other embodiments, for example where the backwards legs are deployed by being released, ring 972 would retract (or an overtube would retract) to release the backwards legs to deploy themselves. In some embodiments, the backward legs are plastically deformed by ring 972, to deploy them.

FIG. 14E is a perspective view showing only some of spikes 105, locked to forward legs 104.

Tear Connector (1624)

In some embodiments of the invention, for example the one pictured herein, the connector used is of a type where part of the connector is torn off of the section which remains in the body. In an exemplary embodiment of the invention, weakened locations are defined on legs 104 and retraction of connector section 102 relative to ring 972 causes these weakened sections to tear.

FIG. 15A shows an anastomosis after tearing is completed.

FIG. 15B is a perspective view of the connector. Actually, the connector comprises a plurality of individual legs 104, each of which has an individual sliding spike 105. A portion 984 of legs 104 is adapted for holding of the legs during retraction. A portion 980 comprises an aperture in the leg, and is a weakening adapted for tearing. A portion 982 comprises a locking section which optionally includes a stop to prevent forward motion of spikes 105 and rearward motion of spikes 105 is optionally prevented by additional stops in leg 104.

Remove Delivery System (1626)

Once the connector is deployed, the delivery system can be removed. In an exemplary embodiment of the invention, system 100 is a splitable system, for example as described in WO 00/56226, the disclosure of which is incorporated herein by reference. As can be noted in substantially all the figures, all the elements show either have a slot or a seam along the axis. In an exemplary embodiment of the invention, body 120 is slid backwards, allowing the various sections to split along the seam. Alternatively or additionally, an active tearing mechanism is used, for example using a knife to cut the system.

In an exemplary embodiment of the invention, section 102 is splitable, so once it is retracted from the tip of delivery system 100 by the retraction mechanism, it can be removed from body 120 and split open. Alternatively, body 120 is split open or allowed to open by removing a restraining pin which keeps it closed. Alternatively, body 120 has a wide slot along it, section 102 can be removed from that slot, once a restraint is removed. In an exemplary embodiment of the invention, the restraint is an outer ring or transverse locking tab which is also coupled to pin 950. When pin 950 is pulled back, the restraint is removed and section 102 can come out of the wide slot in body 120 and split open.

In an alternative embodiment, section 102 is held by an extension of handle 122. Pulling out handle 122, releases section 102. Optionally, a stop is provided inside body 120 to prevent too far a retraction of section 102, which might damage graft 108.

Variations

In an exemplary embodiment of the invention, delivery system 100 is designed for use for attaching a graft to an aorta. The punch may be modified for use with other blood vessels. In addition, different capsules of different connector diameters may be useful for different sizes of target vessels.

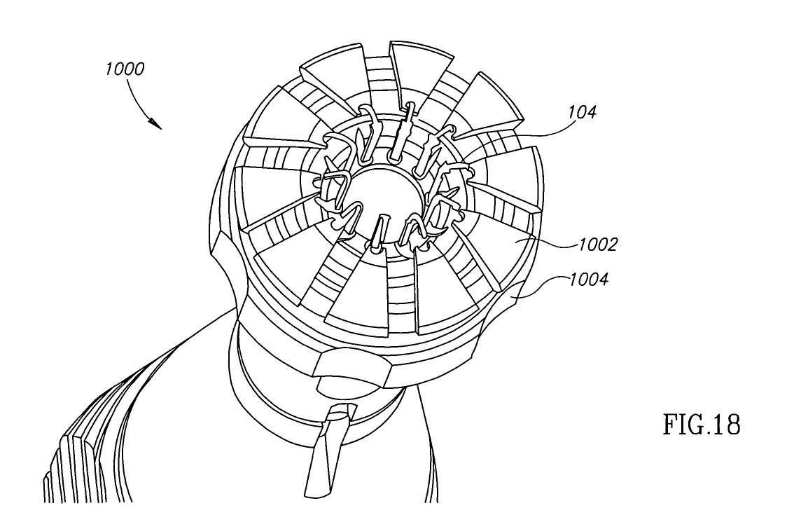

FIG. 18 shows a leg confiner element 1000, in accordance with an exemplary embodiment of the invention. This confiner element may be provided for use with connector section 102, prior to eversion, for example being packaged with it. One potential advantage of confiner element 1000 is that it protects legs 104 and spikes 105 from inadvertent contact and damage. In an exemplary embodiment of the invention, element 1000 includes a body 1004, which, when rotated, selectively moves a plurality of leg compressing elements 1002 radially inwards or outwards, thereby selectively compressing legs 104 together. Such compression may be useful during eversion, to reduce the effective diameter of the connector during eversion. Alternative eversion tools may be used as well, for example, lassos or tweezers where the arms end in small loops.

In an exemplary embodiment of the invention, the pass-by mechanism allows the diameter of the delivery system to remain relatively small, except at the point where the two sections pass by each other. Optionally, this section is made elastic, so that the system can expand at the point for a short time (e.g., which point is or is not in a human body). In some embodiments of the invention, only the penetration tip of the punch is retracted, in which case, the increase to the diameter can be relatively small. In an exemplary embodiment of the invention, the total diameter of the delivery system, at points near the tip (e.g., within 10 cm) and/or at points adapted to be inside the body, are less than 100%, 50%, 30%, 20% or less, greater than a diameter of a deployed connector. In the case of a radially compressible connector, the total diameter of the delivery system may be the same or even less than the diameter of the connector. Optionally, the resting location for the punch section is 1 cm, 3 cm, 5 cm, 10 cm or any intermediate or greater distance from the area of contact with the blood vessel.

Wire Based Embodiment

FIG. 19 schematically illustrates a wire based delivery system 1900, in accordance with an exemplary embodiment of the invention.

Punch section 140 is selectively retracted by a wire 1908 (or flexible rod) which is attached to a loop 1904, for example a wire or a ribbon. Loop 1904 is optionally mounted between a pulley 1906 and a second pulley shown here as being optionally integrated with a knob 1902. When knob 1902 is rotated (or the loop moved in another manner), wire 1908 retracts punch section 140. A base 1910 is mounted on loop 1904 and is also optionally connected to the forward connector legs, as will be described below.

Connector section 102 is mounted, but optionally not attached to loop 1904, for example, a rider section 1912 thereof riding on loop 1904. Rotation of knob 1902 will advance base 1910 and with it connector section 102, for example by base 1910 pushing against a base section 1914 of connector section 102. Once sufficiently advanced, an interlocking between connector section 102 and overtube 132 and/or a body 1924 comes optionally into play, as will be described below. Optionally, the interlocking is by protrusions 1918 of section 102 engaging protrusions 1922 of overtube 132. A matching set of protrusions 1916 of section 102 and 120 of body 1924 slip past each other, as optionally interlock later, as will be described below. Further rotation of knob 1902 is optionally prevented mechanically by section 102 abutting against body 1924. Optionally, protrusion 1916 is coupled to section 102 itself, which thereby couples it to the backwards legs of the connector. Optionally, protrusion 1918 is coupled to base 1910 (e.g., via a slot in section 102).

In deploying the connector, knob 1902 is optionally rotated in an opposite direction. At the beginning, the connector optionally interconnects section 102 and base 1910, so that overtube 132 is pulled back by its interlocking with section 102 and torn (alternatively, base 1910 is coupled via protrusion 1918 to overtube 132 and base section 102 doesn't retract). At the same time, base 1910 pulls back the forward connector legs causing retraction. Backward motion of the backward legs is prevented by interlocking of protrusions 1916 with body 1924, eventually causing tearing of the forward legs and deployment of the device. Optionally, some leeway is provided between protrusions 1916 and 1920, to allow a small amount of retraction of the backwards legs of the connector.

Optionally, a wire and/or pulley based mechanism is used for a delivery system with only one tool (e.g., punch or connector). Optionally, knob 1902 is rotated using a hydraulic actuator, a pneumatic actuator or an electric motor, rather than manually.

A potential advantage of using pull wires is that a punch section can be pulled back a long way using wires, for example by winding wire 1908 on a take-up spool (or around knob 1902), without requiring a corresponding increase in device length. Alternatively or additionally, a pulley based block and tackle mechanism can be used (e.g., inside the body) to obtain a mechanical or length advantage. Optionally, such a block and tackle mechanism provides a length advantage factor of 2, 4, 5, 10 or a smaller intermediate or larger factor. In an exemplary embodiment of the invention, a length advantage is used for a shape memory based mechanism. In such a shape memory based mechanism, the length of extension of a heated NiTi wire may be limited to 5%. By using a pulley system with a block and tackle arrangement, this can be translated into a significant length change, even for a relatively short NiTi wire. In another embodiment, a long NiTi wire is provided wound multiple times (e.g., 2, 3, 4, 5 or more) between two pulleys. The total length change can be considerable for a relatively compact system, which may be provided as a removable capsule. Optionally, such NiTi wire is heated over significant parts of its length, for example 50%, 70% or more.

In an exemplary embodiment of the invention, such a wire based mechanism is used for a system which includes only a connector deployment or only punching, but not both.

Fluid Based Embodiment

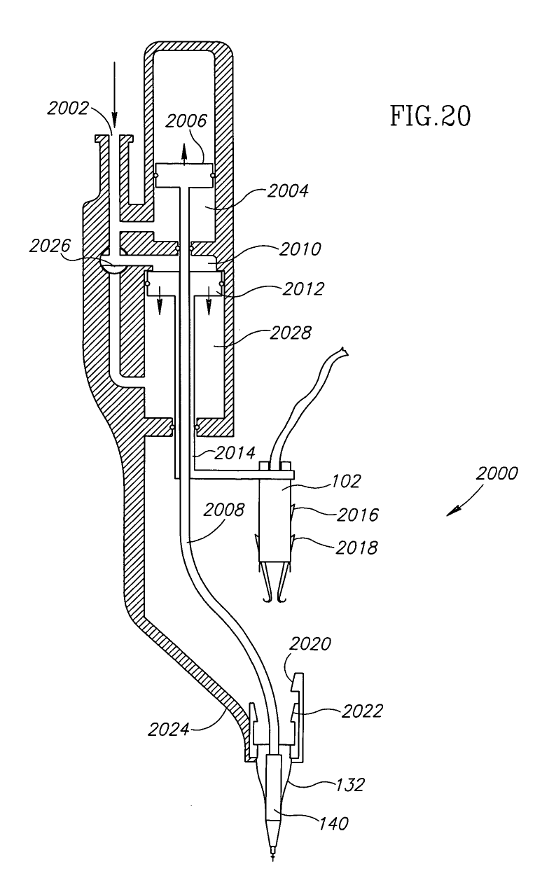

FIG. 20 schematically illustrates a fluid based delivery system 2000, in accordance with an exemplary embodiment of the invention.

Fluid, for example saline, enters a nozzle 2002 and an expanding chamber 2004. A piston 2006 moves proximally from the fluid pressure and pulls a cable or flexible rod 2008 with it. Punch section 140 is pulled along with rod 2008. Optionally, the flexible rod is pre-disposed to move punch section 140 to the side, once it is free of the end of a system body 2024. The same fluid optionally flows through a valve 2026 to an expanding chamber 2010, to push a piston 2012. Piston 2012 is optionally coupled to connector section 102 (optionally only to forward connector legs thereof), via a coupling 2014 and advances section 102 thereby. Optionally, the relative cross-section of the delivery tubes to the chambers or of the chambers themselves is selected so that piston 2006 moves punch section 140 out of the way in time for connector section 102 to arrive.

An interlock between body 2024 and section 102 is optionally provided by sets of protrusions 2016 and 2020. An interlock between the forward connector legs and overtube 132 is optionally provided by sets of protrusions (or other means) 2022 and 2018.

When connector deployment is desired, valve 2026 is rotated, so that a chamber 2028 on an opposite side of piston 2012 fills and retracts piston 2012 and section 102 with it. Optionally, coupling 2014 is attached only to the forward legs, thereby retracting them. Optionally, valve 2026 rotates automatically when piston 2012 stops advancing under increased pressure.

Retraction of the forward legs optionally tears overtube 132 (via protrusions 2022). Section 102 and the backwards legs stay in place, thereby causing eventual tearing of the forward legs and deployment of the device.

Optionally, the fluid is provided using volume control. Optionally, release valves to release pressure are provided, for example to prevent damage to system 2000. Optionally, the expanding chambers are otherwise arranged, for example being side by side or being inline with connector section 102.

Application

The following documents, the disclosures of which are incorporated herein by reference describe connectors, delivery systems and/or other tools and methods which are useful in conjunction with embodiments of the present invention:
PCT/IL03/00774, filed on Sep. 25, 2003, now published as WO 2004/028373;
PCT/IL03/00770, filed on Sep. 25, 2003, now published as WO 2004/028376;
PCT/IL03/00769, filed on Sep. 25, 2003, now published as WO 2004/028377;
PCT/IL03/00959, filed on Nov. 13, 2003, now published as WO 2004/043216;
PCT/IL02/00790, filed on Sep. 25, 2002, now published as WO 03/026475;
U.S. Ser. No. 60/492,998 filed on Aug. 7, 2003;
PCT/IL02/00215, filed on Mar. 18, 2002, now published as WO 02/074188;
PCT/IL01/01019, filed on Nov. 4, 2001, now published as WO 02/47532;
PCT/IL01/00903, filed on Sep. 25, 2001 now published as WO 02/30172;
PCT/IL01/00600, filed on Jun. 28, 2001, now published as WO 02/47561;
PCT/IL01/00267, filed on Mar. 20, 2001, now published as WO 01/70091;
PCT/IL01/00266, filed on Mar. 20, 2001, now published as WO 01/70090;
PCT/IL01/00074, filed on Jan. 25, 2001, now published as WO 01/70119;
PCT/IL01/00069, filed on Jan. 24, 2001, now published as WO 01/70118;
PCT/IL00/00611, filed on Sep. 28, 2000, now published as WO 01/41624;
PCT/IL00/00609, filed on Sep. 28, 2000, now published as WO 01/41623,
PCT/IB00/00310, filed on Mar. 20, 2000, now published as WO 00/56228;
PCT/IB00/00302, filed on Mar. 20, 2000, now published as WO 00/56227;
PCT/IL99/00674, filed on Dec. 9, 1999, now published as WO 00/56223;
PCT/IL99/00670, filed on Dec. 8, 1999, now published as WO 00/56226;
PCT/IL99/00285, filed on May 30, 1999, now published as WO 99/62408; and
PCT/IL99/00284, filed on May 30, 1999, now published as WO 99/62415.

And unpublished application PCT/IL2004/000311, filed on Apr. 4, 2004, in English and designating the US.

In addition, the following US provisional applications, the disclosures of which are incorporated herein by reference, also describe connectors, delivery systems and/or other tools and methods which are useful in conjunction with embodiments of the prevent invention:

U.S. provisional application 60/518,677 filed on Nov. 12, 2003 and U.S. provisional application 60/561,091, filed on Apr. 8, 2004.

A clip or a connector may be manufactured of various materials, including for example, metals (e.g., stainless steel alloys, NiTi alloys and titanium), plastics and bio-absorbable materials. Optionally, the clip is formed of a material that exhibits elastic, super elastic and/or shape memory properties.

Some of these applications describe anastomosis delivery systems and hole making apparatus and/or other device useful in cooperation with the present invention. Some of these applications describe delivery systems in which separate steps are provided for retracting and tearing, and even, in some embodiments, for advancement of legs.

The above described clips and connectors and their use may be varied in many ways. For example, the leg sections may be interconnected before or after the anastomosis, for example, using a flexible element, such as a suture, or a rigid element, such as a metal bar.

In an exemplary embodiment of the invention, the tips that are designed to penetrate blood vessel tissue are sharpened to minimize trauma to the blood vessels, during attaching, and especially to reduce tearing and/or dissection. For example, the tips may be formed to be needle like, so that they have no edges that can tear nearby tissue, except when inserted, tip first. Such forming may be, for example, by electro-polishing.

While the above clips and delivery systems have been described in general for any type of blood vessel, it should be appreciated that particular modifications may be desired for certain vessel types. For example, the aorta is thicker, while a coronary vessel is thinner, thus suggesting different sizes for a punch or for connectors. For example, an aorta may be 3 mm thick, while a coronary vessel may be less than 1 mm thick.

It should be noted that the term “connector” should be construed broadly to include various types of connectors, including one part, two part and multiple part connectors, some of which when deployed, result in a plurality of individual clip-like sections.

The term “eversion”, where used means not only complete eversion of 180 degrees, but also partial eversion or flaring, for example of 90 degrees. Also, in some embodiments, mounting without eversion is provided.

Measurements are provided to serve only as exemplary measurements for particular cases. The exact measurements stated in the text may vary depending on the application, the type of vessel (e.g., artery, vein, xenograft, synthetic graft), size of connector, shape of hole (e.g., incision, round) and/or sizes of vessels involved (e.g., 1 mm, 2 mm, 3 mm, 5 mm, aorta sized).

In some embodiments, one or more of the devices, generally sterilize, described above, are packaged and/or sold with an instruction leaflet, describing the device dimensions and/or situations for which the device should be applied. Also within the scope of the invention are surgical kits comprising sets of medical devices suitable for making anastomotic connections.

It should be appreciated that the above may be varied and still fall within the scope of the invention, for example, by changing the order of steps or by providing embodiments which include features from several described embodiments or by omitting features described herein. Section headings where are provided are intended for aiding navigation and should not be construed to limiting the description to the headings.

When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. An anastomosis and punch delivery system, comprising:
a punch section containing a punch mechanism;
a connector section containing a connector; and
a switching mechanism which pulls said punch mechanism back and replaces it with said connector mechanism.

2. A system according to claim 1, comprising:
a body in which said sections are contained; and
a handle, which when pushed into said body activates said switching.

3. A system according to claim 2, wherein rotating said handle deploys said connector and completes an anastomosis connection.

4. A system according to claim 1, wherein said mechanism comprises at least one wire which pulls said punch section.

5. A system according to claim 1, wherein said mechanism is automatic.

6. A system according to claim 1, wherein said mechanism couples said pulling and said replacing.

7. A system according to claim 1, wherein said switch mechanism is man-powered.

8. A system according to claim 1, wherein said switch mechanism is machine powered.

9. A system according to claim 1, wherein said mechanism comprises a synchronizing mechanism, for synchronizing actions of the system.

10. A system according to claim 1, wherein said system is splittable.

11. A system according to claim 1, wherein said system is adapted to deploy a connector without the system being moved by an operator relative to a blood vessel.

12. A system according to claim 1, wherein said switch mechanism moves at least one of said sections along a non-linear path.

13. A system according to claim 1, wherein said switch mechanism retracts an overtube such that the overtube is torn.

14. A connector control mechanism, comprising:
a shaft;
at least two nuts mounted on said shaft; and
at least two extensions coupled to said nuts and extending in a same direction,
wherein rotating said shaft in one direction causes one of said extensions to extend and one of said extensions to retract relative to said shaft.

15. A connector control mechanism, comprising:
a connector;
an overtube adapted to be placed in an aperture of a blood vessel;
a connector advancing mechanism configured to advance said connector through said overtube; and
an overtube retraction mechanism configured to retract said overtube in synchrony with retraction of said connector, prior to full retraction of said connector.

16. A mechanism according to claim 15, comprising a ring over which said overtube is retracted, said ring having a diameter smaller than that of said overtube.

17. A punch mechanism, comprising:
a cutting tube;
a penetration tip having an axis;
a spring configured to retract said penetration tip relative to said cutting tube; and
a removable pin provided in a direction perpendicular to said axis and coupling said penetration tip to said cutting tube thereby selectively preventing said spring from said retracting.

18. A graft delivery system, comprising:
a body;
an overtube adapted to be placed in a blood vessel;
an overtube retractor; and
an overtube splitter adapted to split said overtube when said retractor retracts said overtube towards said body.

19. A system according to claim 18, wherein said overtube is pre-weakened.

20. A system according to claim 18, wherein said overtube is pre-split.

21. A system according to claim 18, wherein said overtube is adapted to act as a blood vessel cutter.
